(19) United States
(12) Patent Application Publication
(10) Pub. No.: US 2008/0275305 A1
(43) Pub. Date: Nov. 6, 2008

(54) Medical scanned beam imager and components associated therewith

(75) Inventors: Robert J. Dunki-Jacobs, Mason, OH (US); Michael P. Weir, Blanchester, OH (US)

Correspondence Address: THOMPSON HINE L.L.P.;Intellectual Property Group, P.O. BOX 8801, DAYTON, OH, 45401-8801, US

(73) Assignee: Ethicon Endo-Surgery, Inc.

(21) Appl. No.: 11/799,122

(22) Filed: May 1, 2007

(51) Int. Cl. A61B 1/07

(52) U.S. Cl. 600/175

(57) ABSTRACT

A first expression of a first apparatus includes a medical scanned beam imager including an optical dome and a scanner, wherein the optical dome has a variable optical power distribution. A first expression of a second apparatus includes a sleeve assembly which is attachable to a medical scanned beam imager and which includes an objective element which covers an optical dome of the imager when the sleeve assembly is attached to the imager. A second expression of a second apparatus includes a sleeve assembly and a medical scanned beam imager wherein the sleeve assembly is attached to the imager and an objective element of the sleeve assembly covers an optical dome of the imager. A first expression of a third apparatus includes an objective element and a medical scanned beam imager having an optical dome wherein the objective element covers the optical dome and is attached to the imager.

### FIELD OF THE INVENTION

The present invention is related generally to medical equipment, and more particularly to a medical scanned beam imager and to components associated therewith.

### BACKGROUND OF THE INVENTION

An example of an endoscope application of a medical scanned laser beam imager having an optical dome is given in US Patent Application Publication 2005/0020926. The scanned laser beam imager includes a two-dimensional MEMS (micro-electromechanical system) scanner. The MEMS scanner is a dual-resonant-mirror scanner. The mirror scanner scans, about substantially orthogonal first and second axes, one or more light beams (such as light beams from red, green and blue lasers) through an optical dome at high speed in a pattern that covers an entire two-dimensional field of view or a selected region of a two-dimensional field of view. The scanned laser beam imager uses at least one light detector in creating a pixel image from the reflected light for display on a monitor. It is noted that FIG. 1 of US Patent Application Publication 2005/0020926 shows a schematic diagram of a scanned beam imager without an optical dome and that FIGS. 12 and 26 of US Patent Application Publication 2005/0020926 show a non-schematic side-elevational view of a portion of a scanned beam imager including the scanner and the optical dome. The scanned laser beam imaging endoscope of US Patent Application Publication 2005/0020926 may be described as a front-view endoscope.

Conventional (non-scanning) endoscopes include front-view endoscopes and side-view endoscopes.

What is needed is an improved medical scanned beam imager and components associated therewith.

### SUMMARY

A first expression of a first embodiment of the invention is for apparatus including a medical scanned beam imager. The medical scanned beam imager includes an optical dome and a scanner. The scanner is adapted to scan a beam of light through the optical dome. The optical dome has a variable optical power distribution.

A first expression of a second embodiment of the invention is for apparatus including a sleeve assembly for a medical scanned beam imager. The medical scanned beam imager includes a tube having a distal end insertable into a patient and includes an optical dome supported by the tube proximate the distal end of the tube. The sleeve assembly includes a sleeve having a distal end portion and includes an objective element attached to the distal end portion of the sleeve. The sleeve assembly is surroundingly attachable to the tube. The objective element covers and is positioned distal the optical dome when the sleeve is attached to the tube.

A second expression of a second embodiment of the invention is for apparatus including a sleeve assembly and a medical scanned beam imager. The medical scanned beam imager includes a tube having a distal end insertable into a patient and includes an optical dome supported by the tube proximate the distal end of the tube. The sleeve assembly includes a sleeve having a distal end portion and includes an objective element attached to the distal end portion of the sleeve. The sleeve is surroundingly attached to the tube. The objective element covers and is positioned distal the optical dome.

A first expression of a third embodiment of the invention is for apparatus including an objective element and a medical scanned beam imager. The medical scanned beam imager includes a tube having a distal end insertable into a patient and includes an optical dome supported by the tube proximate the distal end of the tube. The objective element covers the optical dome and is attached to the distal end of the tube distal the optical dome.

Several benefits and advantages are obtained from one or more of the embodiments of the invention. In one example of the first embodiment, the medical scanned beam imager displays an image of the upper or lower gastrointestinal tract of a patient which allows the user to use the image portion from a (e.g., zero-optical-power) first region of the optical dome for navigation within the upper or lower gastrointestinal tract and allows the user to use the (e.g., magnified) image portion from a surrounding (e.g., positive-optical-power) second region of the optical dome for identification of pathologies in the wall of the upper or lower gastrointestinal tract.

In one example of the second embodiment, the sleeve assembly is removably attachable or attached to the medical scanned beam imager and is disposable, and different sleeve assemblies having different variable optical power distributions for the objective element may be used with the same reusable medical scanned beam imager. In the same or different example of the second embodiment and/or an example of the third embodiment, the objective element has a (e.g., substantially flat) distal end surface having a smaller total crevice area than the distal end surface of the medical scanned beam imager which makes cleaning or sterilization easier.

In the same or different example of the second and/or third embodiment, the objective element has a circular first region and a surrounding annular second region of different optical power wherein a beam of light from the scanner of the medical scanned beam imager is scanned through the optical dome and through the first region of the objective element, and wherein light returned from the patient passes through the second region of the objective element and is collected by an annular array of optical fibers of the medical scanned beam imager. In one variation, the first region increases or decreases the scanning field of view of the medical scanned beam imager, and the second region functions to extend or modify the pattern of sensitivity of the collecting fibers. In the same or different example of the third embodiment, a standardized medical scanned beam imager is individualized for a particular customer by a manufacturer attaching a customized objective element to the tube of the standardized medical scanned beam imager.

### BRIEF DESCRIPTION OF THE FIGURES

FIG. 1 is a diagram illustrating a first embodiment of the invention which shows a distal portion of a medical scanned beam imager including a scanner and an optical dome;

FIG. 2 is a view of the optical dome of FIG. 1 removed from the medical scanned beam imager of FIG. 1;

FIG. 3 is a view of the optical dome of FIG. 2 taken along lines 3-3 of FIG. 2;

FIG. 4 is a view, as in FIG. 2, but of an alternate embodiment of an optical dome;

FIG. 5 is a view of the optical dome of FIG. 4 taken along lines 5-5 of FIG. 4;

FIG. 6 is a diagram of a second embodiment of the invention including a sleeve assembly and a medical scanned beam imager;

FIG. 7 is a view of the sleeve assembly of FIG. 6 detached from the medical scanned beam imager of FIG. 6;

FIG. 8 is a view of the objective element of the sleeve assembly of FIG. 7, taken along lines 8-8 of FIG. 7; and

FIG. 9 is a diagram of a third embodiment of the invention including an objective element and a medical scanned beam imager.

### DETAILED DESCRIPTION

Before explaining the several embodiments of the present invention in detail, it should be noted that each is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is further understood that any one or more of the following-described embodiments, examples, etc. can be combined with any one or more of the other following-described embodiments, examples, etc.

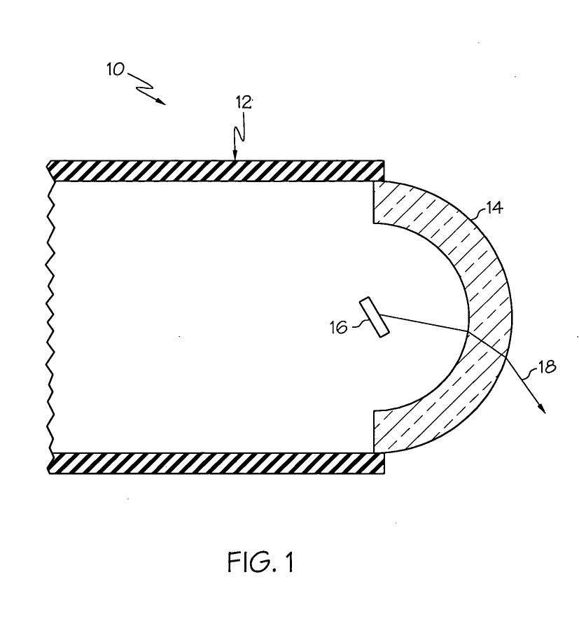

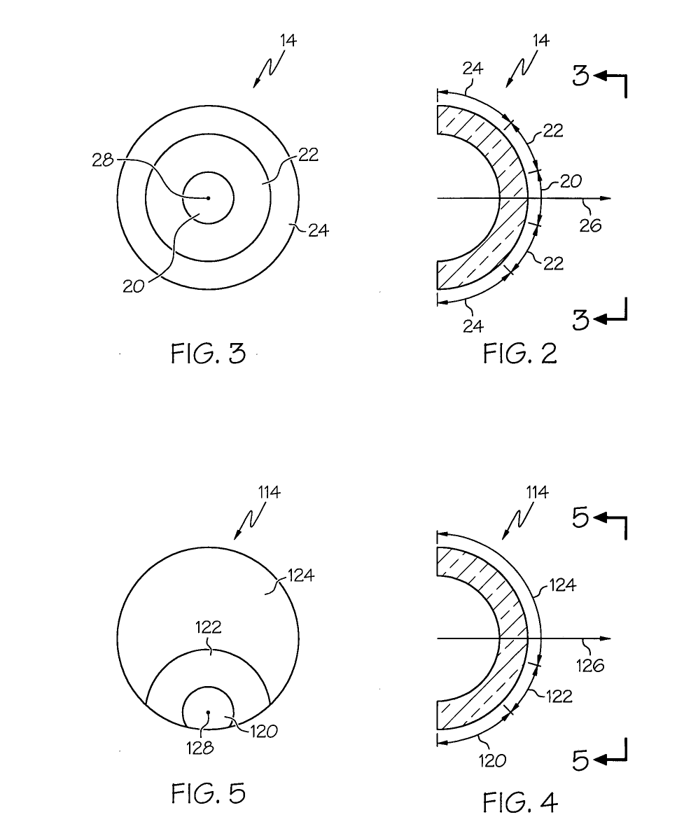

A first embodiment of the invention is shown in FIGS. 1-3. A first expression of the embodiment of FIGS. 1-3 is for apparatus 10 including a medical scanned beam imager 12. The medical scanned beam imager 12 includes an optical dome 14 and a scanner 16. The scanner 16 is adapted to scan a beam of light 18 through the optical dome 14. The optical dome 14 has a variable optical power distribution.

In one construction, not shown, of the first expression of the embodiment of FIGS. 1-3, the medical scanned beam imager 12 also includes a controller operatively connected to the scanner 16, to a light beam source assembly (such as a laser source) and to at least one light detector, wherein the light beam source assembly transmits a light beam through a fiber optic cable to a stationary mirror which reflects the light beam onto the scanner 16. In one variation, the scanner 16 is a dual-resonant-mirror scanner which oscillates about orthogonal first and second axes to scan the beam of light 18 through the optical dome 14 to the patient. It is noted that the term “resonant” means having an angular excursion about the scan axes that is substantially sinusoidal in time and that the term “patient” includes a human patient and a non-human patient. In the same or a different variation, an annular array of fiber optic cables surrounds the circumference of the optical dome 14, receives light returned from the patient, and transmits the returned light to the light detector or detectors. In the same or a different variation, the controller is operatively connected to a monitor, creates an image from the returned light, and displays the image on the monitor. Other constructions are left to those skilled in the art.

In one implementation of the first expression of the embodiment of FIGS. 1-3, the optical dome 14 includes a plurality of regions 20, 22 and 24 each having different optical power. In a first variation, the plurality of regions 20, 22 and 24 each have a constant optical power. Examples of a constant optical power include a constant zero optical power, a constant positive optical power, and a constant negative optical power.

In one elaboration, the plurality of regions 20, 22 and 24 includes a first region 20 and a second region 22, wherein the first region 20 is an at-least-partially-circular region, and wherein the second region 22 is an at-least-partially-annular region and at least partially surrounds the first region 20. In one modification, the first region 20 is a fully circular region and the second region 22 is a fully annular region completely surrounding the first region 20. In one extension, the plurality of regions 20, 22 and 24 includes a third region 24, wherein the third region 24 is an at-least-partially-annular region and at least partially surrounds the second region 22. In one illustration, the third region 24 is a fully annular region completely surrounding the second region 22. In one arrangement, the optical dome 14 has a dome central axis 26, and the first region 20 has a center 28 aligned with the dome central axis 26.

FIGS. 4 and 5 show an alternate embodiment of an optical dome 114 which is similar to the optical dome 14 of FIGS. 1-3. Unlike the optical dome 14 of FIGS. 1-3, in the alternate embodiment of the optical dome 114 of FIGS. 4-5, the first region 120 is a partially-circular region, the second region 122 is a partially-annular region, and the third region 124 is a partially-annular region. Also, unlike the optical dome 14 of FIGS. 1-3, in the alternate embodiment of the optical dome 114 of FIGS. 4-5, the first region 120 has a center 128 which is offset from the dome central axis 126 of the optical dome 114.

Other numbers and configurations of constant or variable optical power regions of the optical dome, including an optical dome whose optical power varies with the surface distance from a reference point on a surface of the optical dome, are left to the artisan.

In one endoscopic employment of the medical scanned beam imager 12, the medical scanned beam imager 12 displays an image (not shown) of the upper or lower gastrointestinal tract of a patient which allows the user to use the image portion from the (e.g., zero-optical-power) first region 20 of the optical dome 14 for navigation within the upper or lower gastrointestinal tract and allows the user to use the (e.g., magnified) image portion from the surrounding (e.g., positive-optical-power) second region 22 of the optical dome 14 for identification of pathologies in the wall of the upper or lower gastrointestinal tract.

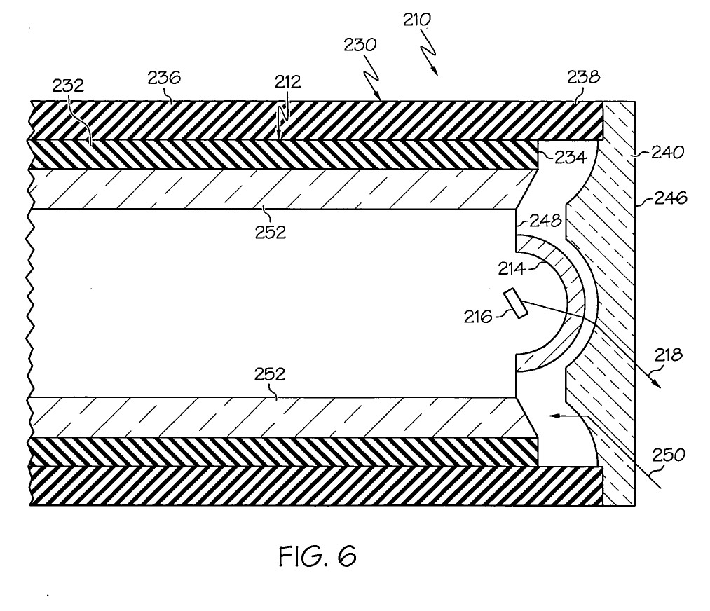

A second embodiment of the invention is shown in FIGS. 6-8. A first expression of the embodiment of FIGS. 6-8 is for apparatus 210 including a sleeve assembly 230 for a medical scanned beam imager 212. The medical scanned beam imager 212 includes a tube 232 having a distal end 234 insertable into a patient and includes an optical dome 214 supported by the tube 232 proximate the distal end 234 of the tube 232. The sleeve assembly 230 includes a sleeve 236 having a distal end portion 238 and includes an objective element 240 attached to the distal end portion 238 of the sleeve 236. The sleeve assembly 230 is surroundingly attachable to the tube 232. The objective element 240 covers and is disposed distal the optical dome 214 when the sleeve 236 is attached to the tube 232.

It is noted that in the first expression of the embodiment of FIGS. 6-8, the objective element 240 and/or the optical dome 214 may have a constant zero optical power distribution, a constant positive optical power distribution, a constant negative optical power distribution, or a variable optical power distribution.

In one enablement of the first expression of the embodiment of FIGS. 6-8, the objective element 240 has a variable optical power distribution. In one variation, the objective element 240 includes a first region 242 and a second region 244, wherein the first region 242 is a fully-circular region, wherein the second region 244 is a fully-annular region completely surrounding the first region 242, and wherein the first and second regions 242 and 244 each have different optical power. In one modification, the first region 242 has a constant optical power and the second region 244 has a constant optical power. It is noted that the discussion of optical power regions of the optical dome 14 and 114 of the embodiments of FIGS. 1-5 is equally applicable to optical power regions for the objective element 240 of the first expression of the embodiment of FIGS. 6-8.

A second expression of the embodiment of FIGS. 6-8 is for apparatus 210 including a sleeve assembly 230 and a medical scanned beam imager 212. The medical scanned beam imager 212 includes a tube 232 having a distal end 234 insertable into a patient and includes an optical dome 214 supported by the tube 232 proximate the distal end 234 of the tube 232. The sleeve assembly 230 includes a sleeve 236 having a distal end portion 238 and includes an objective element 240 attached to the distal end portion 238 of the sleeve 236. The sleeve 236 is surroundingly attached to the tube 232. The objective element 240 covers and is disposed distal the optical dome 214.

It is noted that the enablements, etc. of the first expression of the embodiment of FIGS. 6-8 are equally applicable to the second expression of the embodiment of FIGS. 6-8.

In one implementation of the embodiment of FIGS. 6-8, the sleeve assembly 230 is removably attachable or attached to the tube 232 meaning that the sleeve assembly 230 can be detached from the tube 232 without damage to the sleeve assembly 230 or the tube 232. In a different implementation, the sleeve assembly 230 is permanently attachable or attached to the tube 232 meaning that the sleeve assembly 230 cannot be detached from the tube 232 without damage to the sleeve assembly 230 or the tube 232. In one deployment of the embodiment of FIGS. 6-8, an optical transmission medium is disposed between the optical dome 214 and the objective element 240.

In one example of the embodiment of FIGS. 6-8, the sleeve assembly 230 is removably attachable or attached to the tube 232 and is disposable, and different sleeve assemblies having different variable optical power distributions for the objective element 240 may be used with the same reusable medical scanned beam imager 212. In the same or a different example, the objective element 240 has a (e.g., substantially flat) distal end surface 246 having a smaller total crevice area than the distal end surface 248 of the medical scanned beam imager 212 which makes cleaning or sterilization easier. In the same or different example, the objective element 240 has a circular first region 242 and a surrounding annular second region 244 of different optical power wherein a beam of light 218 from the scanner 216 of the medical scanned beam imager 212 is scanned through the optical dome 214 and through the first region 242 of the objective element 240, and wherein light 250 returned from the patient passes through the second region 244 of the objective element 240 and is received by an annular array of fiber optic cables 252 of the medical scanned beam imager 212. In one variation, the first region 242 increases or decreases the scanning field of view of the medical scanned beam imager 212, and the second region 244 functions to extend or modify the pattern of sensitivity of the fiber optic cables 252.

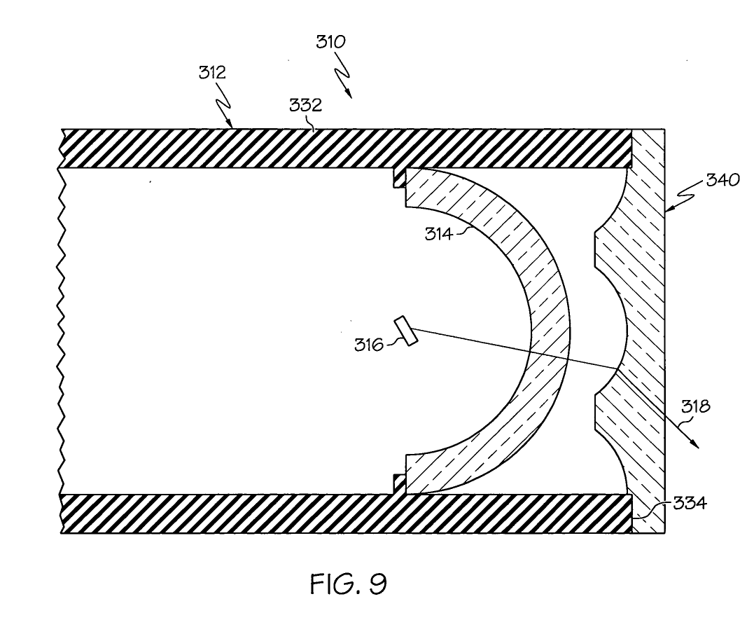

A third embodiment of the invention is shown in FIG. 9. A first expression of the embodiment of FIG. 9 is for apparatus 310 including an objective element 340 and a medical scanned beam imager 312. The medical scanned beam imager 312 includes a tube 332 having a distal end 334 insertable into a patient and includes an optical dome 314 supported by the tube 332 proximate the distal end 334 of the tube 332. The objective element 340 covers the optical dome 314 and is attached to the distal end 334 of the tube 332 distal the optical dome 314.

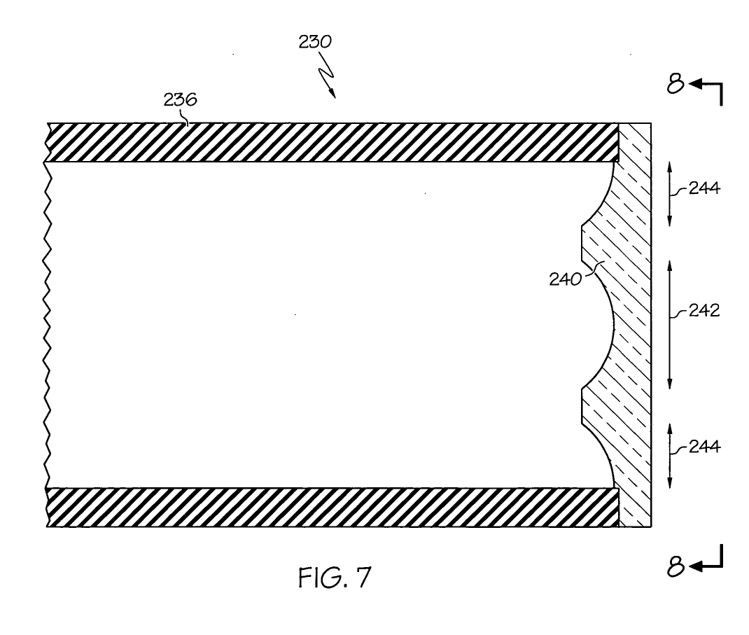

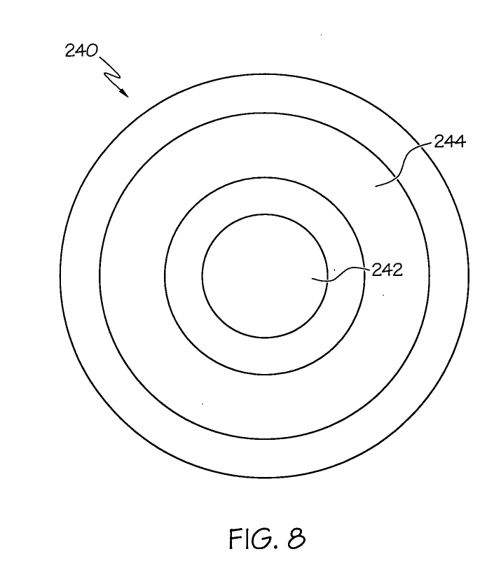

It is noted that the discussion of the optical power of the objective element 240 of the embodiment of FIGS. 6-8 is equally applicable to the objective element 340 of the embodiment of FIG. 9. In one employment, objective element 340 is identical to objective element 240, wherein first and second regions of objective element 340 are identical to the first and second regions 242 and 242 of the objective element 240 as shown in FIGS. 7 and 8.

In one employment of the first expression of the embodiment of FIG. 9, a standardized medical scanned beam imager 312 is individualized for a particular customer by a manufacturer attaching a customized objective element 340 to the tube 332 of the standardized medical scanned beam imager 312, wherein a beam of light 318 from the scanner 316 of the medical scanned beam imager 312 is scanned through the optical dome 314 and through the objective element 340.

While the present invention has been illustrated by a description of several expressions of embodiments, it is not the intention of the applicants to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. Apparatus comprising a medical scanned beam imager, wherein the medical scanned beam imager includes an optical dome and a scanner, wherein the scanner is adapted to scan a beam of light through the optical dome, and wherein the optical dome has a variable optical power distribution.

2. The apparatus of claim 1, wherein the optical dome includes a plurality of regions each having different optical power.

3. The apparatus of claim 2, wherein the optical power is constant in each region.

4. The apparatus of claim 3, wherein the plurality of regions includes a first region and a second region, wherein the first region is an at-least-partially-circular region, and wherein the second region is an at-least-partially-annular region and at least partially surrounds the first region.

5. The apparatus of claim 3, wherein the plurality of regions includes a fully circular region and a fully annular region completely surrounding the fully circular region.

6. The apparatus of claim 5, wherein the optical dome has a dome central axis, and wherein the first region has a center aligned with the dome central axis.

7. The apparatus of claim 4, wherein the optical dome has a dome central axis, and wherein the first region has a center offset from the dome central axis.

8. Apparatus comprising a sleeve assembly for a medical scanned beam imager, wherein the medical scanned beam imager includes a tube having a distal end insertable into a patient and includes an optical dome supported by the tube proximate the distal end of the tube, wherein the sleeve assembly includes a sleeve having a distal end portion and includes an objective element attached to the distal end portion of the sleeve, wherein the sleeve assembly is surroundingly attachable to the tube, and wherein the objective element covers and is disposed distal the optical dome when the sleeve is attached to the tube.

9. The apparatus of claim 8, wherein the objective element has a variable optical power distribution.

10. The apparatus of claim 9, wherein the objective element includes a first region and a second region, wherein the first region is a fully-circular region, wherein the second region is a fully-annular region completely surrounding the first region, and wherein the first and second regions each have different optical power.

13. The apparatus of claim 10, wherein the first region has a constant optical power and wherein the second region has a constant optical power.

14. Apparatus comprising a sleeve assembly and a medical scanned beam imager, wherein the medical scanned beam imager includes a tube having a distal end insertable into a patient and includes an optical dome supported by the tube proximate the distal end of the tube, wherein the sleeve assembly includes a sleeve having a distal end portion and includes an objective element attached to the distal end portion of the sleeve, wherein the sleeve is surroundingly attached to the tube, and wherein the objective element covers and is disposed distal the optical dome.

15. The apparatus of claim 14, wherein the objective element has a variable optical power distribution.

16. The apparatus of claim 15, wherein the objective element includes a first region and a second region, wherein the first region is a fully-circular region, wherein the second region is a fully-annular region completely surrounding the first region, and wherein the first and second regions each have different optical power.

17. The apparatus of claim 16, wherein the first region has a constant optical power and wherein the second region has a constant optical power.

18. Apparatus comprising an objective element and a medical scanned beam imager, wherein the medical scanned beam imager includes a tube having a distal end insertable into a patient and includes an optical dome supported by the tube proximate the distal end of the tube, and wherein the objective element covers the optical dome and is attached to the distal end of the tube distal the optical dome.

19. The apparatus of claim 18, wherein the objective element has a variable optical power distribution.

20. The apparatus of claim 19, wherein the objective element includes a first region and a second region, wherein the first region is a fully-circular region, wherein the second region is a fully-annular region completely surrounding the first region, and wherein the first and second regions each have different optical power.
